(19) United States
(12) Patent Application Publication
(10) Pub. No.: US 2024/0035015 A1
(43) Pub. Date: Feb. 1, 2024

(54) SYSTEM AND METHOD FOR CONTROLLING CELLULAR ADHESION WITH THE AID OF A DIGITAL COMPUTER

(71) Applicant: XEROX CORPORATION, NORWALK, CT (US)

(72) Inventors: Krishnan Thyagarajan, Sunnyvale, CA (US); Christopher Somogyi, Wimberley, TX (US)

(21) Appl. No.: 17/876,072

(22) Filed: Jul. 28, 2022

(51) Int. Cl. C12N 13/00; C12M 1/12; C12M 1/34; C12M 1/26

(52) U.S. Cl. CPC C12N 13/00; C12M 25/14; C12M 41/46; C12M 33/00

(57) ABSTRACT

A feedback system that identifies characteristics of one or more cells being grown on a metasurface and utilizes the characteristics to initiate and adjust a field applied to the metasurface to control adhesion of the cells to and from the metasurface. The metasurface includes a plurality of structures whose resonances (localized or non-localized) have a wavelength range of 250 nm-3 microns. In one embodiment, the system leverages machine learning to automatically identify characteristics of the one or more cells or the metasurface and adjust the parameters of the field based on the characteristics. Sensors are utilized during the application of the field to monitor characteristics of the one or more cells or the metasurface and parameters of the field. Based on the measurements, parameters of the field can be adjusted to achieve the desired effect on the detachment of the cells.

### FIELD

This application relates in general to adhesion control, and in particular, to a system and method for controlling cellular adhesion with the aid of a digital computer.

### BACKGROUND

As the human population grows, the demand for safe and nutritious food similarly increases, fueling research in food production. In particular, there has been a rapid growth in lab-grown artificial food industry aimed at production of substitutes for terrestrial and aquatic meat products as well as other lab-grown material, including plant-based food. One of the key challenges encountered in lab-based material production is separating the produced material from the surface on which the cells making up the material were grown. For example, mammalian stem cells used in lab-grown meat production produce better output when adhered to a surface. Most cultured meat production setups use a surface such as tiny glass beads (referred to as ‘carriers’) for the cells to adhere to when they are floating about freely in suspension. Adhering to the glass bead surface decreases the amount of physiological stress that the cells experience and provides the cells with access to nutrients, thereby improving the yield and nutrient quality of the produced lab meat. However, after the meat growth reaches a desired stage, the meat of needs to be separated from the surface, either to be packaged for subsequent consumption or to undergo additional processing. Causing this separation is difficult due to the cells' non-stressed state when adhered to the surface, which favors the cells remaining adhered to the surface. Other lab-grown materials can experience similar detachment challenges.

Current techniques for causing the separation of lab-grown material from the surface on which the cells are grown generally employ chemical agents, such as hydrogen peroxide. Such chemical agents are used to destroy the lowest layer of the cells of the lab-grown material that is in contact with the surface; the destruction of the lowest layer in turn causes the remaining portion of the lab-grown material to separate from the surface. However, such techniques decrease the yield of the material, such as meat or other food, recovered due to the destruction of the lowest cell layer. Further, the chemical agents can negatively affect the taste of the recovered material as well the material's nutritional quality. In addition, as different kinds of lab-grown materials exist and the material can be grown in different conditions, the use of chemical agents that produce satisfactory results for certain kinds cells being grown in certain conditions may not be suitable for another strain of cells grown in different conditions. Finally, once the chemical agents are introduced into the medium in which the lab-grown cells are located, their effect cannot easily be modulated if undesirable effects appear and they will continue their action until a chemical equilibrium is reached.

Accordingly, there is a need for a way to control adhesion of lab-grown cells from the surface on which they are grown in a way that conserves the yield of the cells, is specific to the kind of cells whose adhesion is being caused, and whose effect can be adjusted if the adhesion is not proceeding as desired.

### SUMMARY

A feedback system that identifies characteristics of one or more cells being grown on a metasurface and utilizes the characteristics to initiate and adjust a field applied to the metasurface to control adhesion (including attachment and detachment) of the cells to and from the metasurface. The metasurface includes a plurality of structures whose resonances (localized or non-localized) have a wavelength range of 250 nm-3 microns. The application of the field to the desired portions of the metasurface causes the detachment of the cells from those portions of the metasurface while the absence of the field allows the cells to attach to the surface. In one embodiment, the system leverages machine learning to automatically identify characteristics of the one or more cells or the metasurface and adjust the parameters of the field based on the characteristics. Further, if metasurface is part of a larger container, the parameters of the field can further be set and adjusted based on characteristics of parts of the container other than the metasurface as well as characteristics of the medium in which the cells are. Sensors are utilized during the application of the field to monitor characteristics of the one or more cells, the metasurface, the container, the medium, and parameters of the field. Specifically, characteristics of at least some of the cells, the metasurface, or both the cells and the metasurface are measured at different time points and the measurements are used to determine whether the desired effect has been achieved or whether any unintended consequences are taking place. Based on the measurements, parameters of the field can be adjusted to achieve the desired effect on the detachment of the cells. Parameters can further be adjusted based on the characteristics of the container and the medium measured at the different time points. The disclosed system and method allow to control the detachment of specific portions of the artificially-grown material, such as meat or another food, at a specified time without significant loss in yield or material nutritional qualities and while accounting for both the specifics of the cells being grown and any undesirable effects taking place during the detachment. The spatial and temporal control over the detachment further allows to control over the shape of the lab-grown material being produced, with the detachment of the cells at desired locations allowing to stack up the material in the desired locations.

In one embodiment, a system and method for controlling cellular detachment with the aid of a digital computer is provided. A metasurface on which cells of an artificially-grown meat are located is obtained, wherein the metasurface includes a plurality of structures associated with resonances that have a wavelength range of 250 nm-3 microns, wherein the resonances are one of localized or non-localized. Characteristics associated with the cells at one or more spatial locations of the metasurface at multiple time points and characteristics associated with the metasurface at the spatial locations at multiple time points are obtained via one or more sensors. Parameters of at least one field to be applied to the metasurface via at least one field generator are determined based on the metasurface characteristics and cell characteristics determined at the multiple time points. Application of the at least one field via the at least one field generator to at least some of the structures of the metasurface is controlled based on the parameters, wherein the cells attached to those structures disengage from the structures due to the application of the field.

Still other embodiments of the present invention will become readily apparent to those skilled in the art from the following detailed description, wherein is described embodiments of the invention by way of illustrating the best mode contemplated for carrying out the invention. As will be realized, the invention is capable of other and different embodiments and its several details are capable of modifications in various obvious respects, all without departing from the spirit and the scope of the present invention. Accordingly, the drawings and detailed description are to be regarded as illustrative in nature and not as restrictive.

### BRIEF DESCRIPTION OF THE DRAWINGS

FIG. 1 is a block diagram showing a system for controlling cellular adhesion with the aid of a digital computer in accordance with one embodiment.

FIG. 2 is a flow diagram showing a method for controlling cellular adhesion with the aid of a digital computer in accordance with one embodiment.

FIG. 3 is a block diagram showing a top view of a device for feedback-based field application for use in the system and method for controlling cellular adhesion in accordance with one embodiment.

FIG. 4 is a diagram showing, for purposes of illustration and not limitation, a portion of the metasurface that includes three bow-tie shaped motifs in accordance with one embodiment.

### DETAILED DESCRIPTION

Control over cellular adhesion (detachment and attachment) from and to a metasurface on which the cells are grown can be accomplished through application of an electric, electromagnetic, or magnetic fields to the metasurface, monitoring the effects of the applied fields, and adjusting the application of the field as necessary for achieving the detachment of cells at desired portions of the metasurface in a monitoring-based feedback loop. Absence of the field allows the cells to remain attached or reattach to the metasurface. In the description below, the terms “lab-grown material,” “artificially-grown material,” or simply “material” are used interchangeably to refer to a group of cells being artificially grown and can include artificially grown food, such as lab-grown meat or lab-grown plant-based food, though other kinds of artificially-grown materials are also possible.

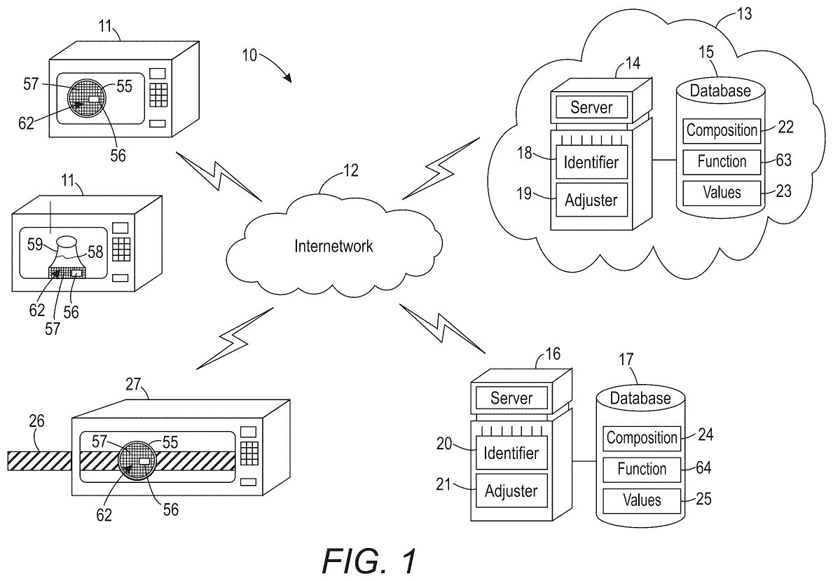

Utilizing a feedback system during application of the fields helps maintain the progression toward the desired result. FIG. 1 is a block diagram showing a system 10 for controlling cellular with the aid of a digital computer in accordance with one embodiment. A field application device 11, 27 can initiate detachment of cells that are part of a lab-grown material 56 grown on a metasurface 62. The metasurface 62 is a nano-textured surface that includes a plurality of structures 57 whose resonances, in one embodiment, are in the range of 250 nm-3 microns, though in a further embodiment other resonances of the structures are possible. The resonances can be localized or non-localized.

The structures 57 can form one or more motifs, such as a bow-tie shaped motif, a coil-shaped motif, a half-coil shaped motif, or a ribcage-shaped motif, though other kinds of motifs are still possible. One metasurface 62 could include multiple repeating motifs of the same shape, or can include multiple motifs of different shapes. FIG. 4 is a diagram showing, for purposes of illustration and not limitation, a portion of the metasurface 62 that includes three bow-tie shaped motifs 81 in accordance with one embodiment. However, as described above, other motifs 81 and other combination of motifs 81 can be a part of the metasurface 62.

The metasurface 62 can be made of metals or dielectrics, though other materials are also possible. In one embodiment, the metasurface 62 can be an integral part of a container in which the cells that are part of the material 56 are grown. For example, such container can be a petri dish 55, or another type of a laboratory glassware such a flask 59, though still other types of containers are possible. Alternatively, the metasurface 62 can be coated on the surface of the containers 55, 59 in which the artificial material 56 is being grown. While with reference to FIG. 1 only the bottom of the containers 55, 59 is shown as including the metasurface 62, in a further embodiment, other portions of the containers 55, 59 such as the sides, could include the metasurface 62. In a still further embodiment, the cells of the material 56 could be grown on a flat or substantially flat metasurface 62 that is not enclosed on all sides and that does not function as a container.

In one embodiment, the containers 55, 59 can be within the field application device 59 while the growth and detachment of the cells 56 occurs. For example, the growth of the cells 56 can further be influenced by the application of the field by the field application device 11, 27, as further described in commonly-assigned U.S. Patent Application, entitled “System and Method for Controlling Cell Functioning and Motility with the Aid of a Digital Computer,” Ser. No. ______, filed Jul. 28, 2022, pending, which is hereby incorporated by reference. In a further embodiment, the cells can be grown outside of the container 55, 59, and brought to into the field application device 27 on a conveyor belt 26 (or another transportation device) as part of the processing in a process-line that the lab-grown material 56 undergoes upon reaching the desired growth stage. The material 56 can be covered by a liquid growth medium 58 within the containers 55, 59 from which the cells of the material 56 extract nutrients necessary for growth, though other kinds of sources of nutrients are also possible. While the material 56 is shown to be of a rectangular shape, other kinds of shapes of the material 56 are also possible. The conveyor belt 26 can be controlled by the processor of the field application device 11, 27 or a different controller.

The application of the field by field generators of the field application device 11, 27 to the portion of the metasurface 62 creates stress in the cells of the material 56 grown on that portion, which in turn causes the detachment of the cells from the portion to which the field is applied. As further described below, the field generators can include electrode, a magnet, wires, electromagnets, two-dimensional conductive material, organic conductive polymer, as well as light sources such as a halogen lamp, a laser, and an LED, though still other kinds of field generators are possible. In one embodiment, when the field applied to the portions of the structures 57 of the metasurface 62 includes light, the structures 57 highly localize the field impinged on them near the surface, thereby producing heat locally, thus affecting the cells attached to those structures or proximate to those structures. The generated heat, thereafter, induces the cells to want to detach from the surface, allowing the cells to be separated on demand. Other mechanisms of causing the cells of the lab-grown material 56 to detach from the surface are also possible. Due to the localized nature of the effect of the field, in one embodiment, a spatial resolution of specificity with which detachment of cells from the metasurface 62 can be caused is 10-20 nm, though in a further embodiment, other kinds of specificity is also possible.

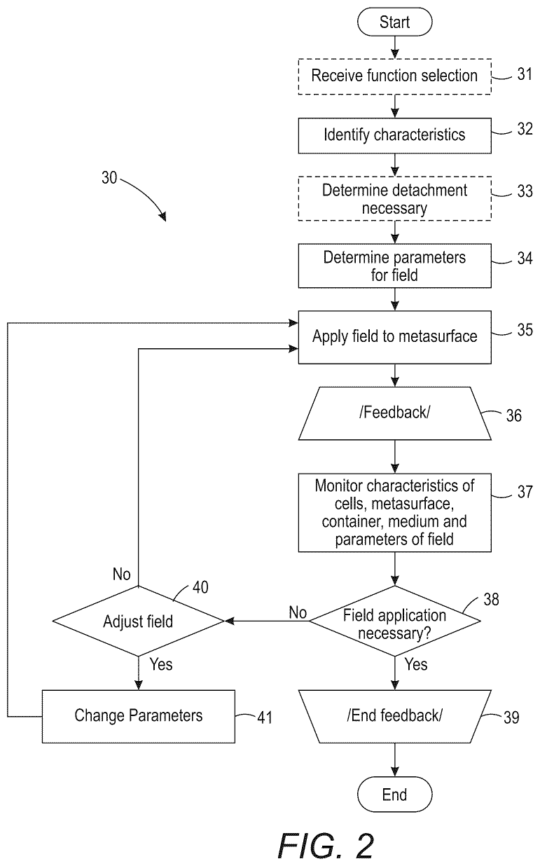

The field is specifically tailored based on identity and characteristics of the cells whose detachment is being caused, such as the kind of organism the cells originate from and the shape and thickness of the meat 56 that the cells form, though other characteristics are also possible. The field is further tailored to the characteristics of the metasurface 62 on which the cells are grown, such as the kind of materials the metasurface 62 is made of, the kind of and location of structures 57 present on the metasurface 62, and the motifs formed at different portions of the metasurface 62 by the structures 57, though still other characteristics of the metasurface 62 are possible. In a further embodiment, the field can be further tailored to characteristics of portions of the container 55, 59 other than the metasurface 62, such as thickness, temperature, and materials of the walls of the containers 55, 59 through which the field needs to be able to pass prior to reaching the metasurface 62, as well as characteristics of the medium 58 the cells are grown in such as optical characteristics, temperature, conductivity, and presence and concentration of nutrients and waste products, though still other characteristics of the containers 55, 59 and the medium 58 are possible. The field is further adjusted based on continued monitoring of the characteristics of the cells and the metasurface 62, and the field (and optionally the container 55, 59 and the medium 58) using the sensors in the device 11, 27 to control progression towards a desired result—this monitoring-based feedback loop is described in detail with reference to FIG. 2. The sensors in the device 11, 27 can also be used to determine when the appropriate time for causing the detachment is, such as through measuring the density of the cells in the medium 58 that are not adhered to the metasurface 62, the concentration of waste products in the medium 58, or a change in the rate of nutrient uptake from the medium 58, though other markers that at least a portion of the meat 56 is ready to be detached are possible. Likewise, after the application of the field has started, the sensors can be used to obtain parameters of the field being applied. The field application device 11, 27 can be a standalone device or can be incorporated into another structure, such as a structure that is a part of a process-line.

The device 11, 27 communicates with a feedback server 14, 16 via an internetwork 12, such as the Internet or a cellular network, to obtain and adjust parameters of the field based on the obtained characteristics. In one embodiment, the feedback server 14 can be a cloud-based server. Alternatively, the server 16 can be locally or remotely located with respect to the field application device 11, 51, 57. The feedback server 14, 16 can include an identifier 18, 20 and an adjuster 19, 21. The identifier 18, 20 can utilize measurements 23, 25 for characteristics of the cells in the meat 56, the metasurface 62, the container 55, 59, and the medium 58 obtained from the field application device 11, 27 to determine an identity or classification of the cells (either individually or the classification of the lab-grown material 56 being grown) based on known composition values 22, 24 of objects stored in a database 15, 17 associated with the server 14, 16. Likewise, the measurements 23, 25 can be used to identify or classify the metasurface 62, the container 55, 56, and the medium 58. Machine learning can also be used in lieu of or in addition to a look up table of compositions and identities or classifications.

The measurement values 23, 25 can further include values for the parameters of the field being applied. The adjuster 19, 21 utilizes measurement values 23, 25 obtained from the field application device 11, 51, 57 to determine an initial set of parameters for the field to be applied and whether the field should be adjusted to ensure an appropriate effect is reached. The adjustment can be determined using characteristic values 23, 25 of cells of the material 56, metasurface 62, container 55, 59, medium 58 and field parameters stored by the databases 15, 17 to determine new parameters for the field. In a further embodiment, ranges of characteristics of the cells of the material 56, metasurface 62, container 55, 59, medium 58 and field parameters can be stored on the device 11, 27 for use in adjusting the fields applied to the cells. Alternatively, machine learning can also be used to determine and adjust field parameters in lieu of a stored look up table of characteristic values and parameters. If different kinds of fields are applied by the same device at the same time, such as if different field generators are used to apply different fields to cells in different portions of the material 56, the initial sets of parameters and the adjustments could be determined for all the fields at the same time or at different times.

A user can specify the portions of the material 56 that needs to detach from the metasurface 62 at a particular time as a function 63, 64 of the field application device 11, 27. The adjuster 19, 21 can utilize the function 63, 64 selected for generation of the parameters. In particular, the function 63, 64 can specify specifically which cells or group of cells in material 56 need to be detached using the field, such as by selecting which field generator included in an array within the device 11, 27 is used to be used to apply the field, as further described with reference as in commonly-assigned U.S. Patent Application, entitled “System and Method for Metamaterial Array-Based Field-Shaping,” Ser. No. ______, filed Jul. 28, 2022, pending, the disclosure of which is incorporated by reference. The function 63, 64 can be entered by the user through the user interface of the device 11, 27 and then wirelessly provided to the adjuster 19, 21, or can be provided to the adjuster 11, 27 through a further computing device, such as a mobile phone or a personal computer interfaced to the adjuster 19, 21 through the Internetwork 12. By controlling the timing of the detachments of different portions of the meat 56, the system 10 allows to further define the shape of the material 56 due to stacking up the different portions.

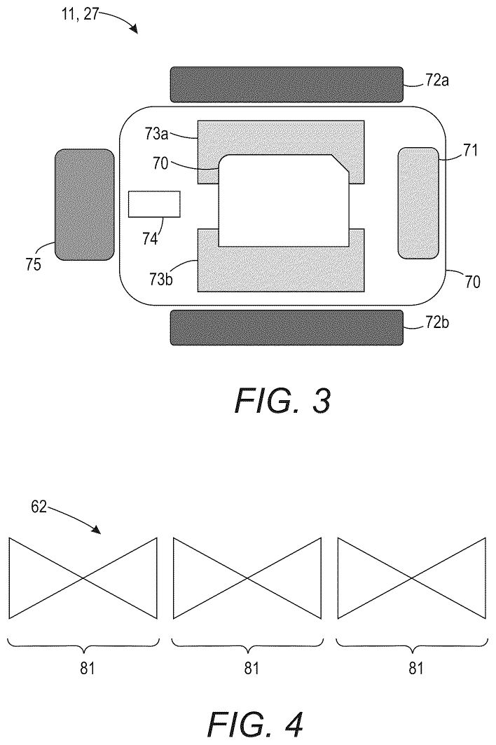

In a further embodiment, identification or classification of the cells of the material 56, the metasurface 62, the container 55, 59, the medium 58, as well as the determination of the parameters of the field can occur on the field application devices 11, 27 such as via a processor, which is described in detail below with respect to FIG. 3.

The ability to automatically determine characteristics of the material 56, the metasurface 62, the container 55, 59, the medium 58, and the field allows to determine and adjust parameters for field application as necessary to maintain progression towards the desired effect. FIG. 2 is a flow diagram showing a method 30 for controlling cellular adhesion with the aid of a digital computer in accordance with one embodiment. The method 30 can be implemented using the system 10 of FIG. 1. A metasurface 62 with cells of material 56 being grown on the metasurface 62 is placed within the field application device 11, 27, either manually or via a conveyor belt 26. The metasurface 62 can be a part of a larger container 55, 59, or can be a standalone structure. Optionally, if the device 11, 27 can accomplish perform multiple kinds of functions that accomplish detachment of cells in a variety of ways, a selection of the function 63, 64 is received (step 31), either into the device 11, 27 in which the cells are positioned, or into the adjuster 19, 21 via the further communication device. The received function 63, 64 can specify that the entire piece of the material 56 needs to be detached from the metasurface 62, either at substantially the same time or with different portions of the artificially-grown meat 56 detaching at different times. Alternatively, the metasurface 62 can include the regions of the material 56 where detachment needs to occur and the timing of such detachment, though other kind of information can be included as part of the function 63, 64. For example, a user may decide to cause detachment of corners of the material 56 before other portions of the meat 56.

A composition or particular characteristics of one or more at least some the cells of the 56, the metasurface 62, the containers 55, 59, and the medium 58, structures formed by those cells are be identified (step 32) via sensors. For example, one or more sensors can send signals towards the cells and information about the cells (and possibly the organisms from which the cells originate) is obtained via the signal, which is returned back to the sensor, and a combination of these sensors, thus obtaining values 23, 25 for the characteristics. Similarly, the values 23, 25 can be obtained for the metasurface 62, the containers 55, 59, and the medium 58. Passive and active sensors can be used, including imaging and reflective sensors, as well as electrocurrent sensors, biosensors, volatile gas sensors, optical sensors (including a camera), chemical sensors, electrochemical sensors, acoustic sensors, and hyperspectral imaging. Likewise, if cell sampling is necessary for a sensor to be used, such as for labeling cells with fluorescent markers when a sensor employs microfluidics techniques, such as flow cytometry, to determine characteristics of the cells, the sensor would be supplemented by automated manipulators (not shown) included in the device 11, 27 necessary for the sampling, the labeling, and presentation of the labeled cells for analysis by the sensors. If the sampling is used, the characteristics of the sampled cells are assigned to cells proximate to where the sampled cells were prior to their removal for analysis by the sensors.

The characteristics of all or only some of the cells that are part of the meat 56 can be determined. The determination of which cells are selected for determination of the characteristics can be based on the selected function 63, 64, though other ways for the device 11, 27 to make the selection are possible.

Further, the characteristics of individual cells could be combined to obtain characteristics of the structures that the cells form, such a shape and dimensions of the meat 56 formed by the cells. For example, the sensors may be able to determine that the meat 56 is rectangular-shaped, 5 inches by 3 inches in dimensions, and has a 1 inch thickness.

Measures 23, 25 for additional characteristics, such as water content, fat content, density, positions of the cells, cell surface roughness, as well as other characteristics, can also be obtained via the sensors. For example, positions of a cell or a group of cells in the material 56 can be obtained via a camera or another optical sensor, and monitored as the cells detach from the metasurface 62.

In one embodiment, the identified characteristics can be used to classify the cells of the meat 56, the metasurface 62, the container 55, 59, and the medium 58. A classification can group cells to an organism from which they originate, such as bovine stem cells or salmon stem cells. Likewise, the metasurface 62 can be classified as being made off a particular metal and with a certain distribution of the structures 57 with specific resonances. Similarly, the container 55, 59 can be classified as being of a particular shape, such as a round petri dish. Similarly, the medium 58 can be classified as a particular kind of a growth medium.

Likewise, classification or identification of an object (such as the cells, the metasurface 62, the container 55, 59, and the medium 58) can occur via a camera or lensless digital holography, using a look up table, be provided by a user, or determined via machine learning. When used, a camera can obtain an image of the object that can be compared with a database of images to determine an identity of the object. The look up table can include characteristics, values for the characteristics, and identities or categories for the object based on the identified characteristics and values.

If user provided, the user can enter values 23, 25 the characteristics and classifications of the cells of the material 56, the metasurface 62, the medium 58, and the container 55, 59 into the field application device 11, 58 or through a further computing device into the adjuster 19, 21. Alternatively, if only the values 23, 25 for the characteristics are provided, machine learning can be used to classify the cells of the material 56, the metasurface 62, the container 55, 59, and the medium 58.

Optionally, if the command to start the detachment is not manually input and is not provided as part of the function 63, 64, the adjuster 19, 21 or the field application device 11, 27 determines that detachment of the cells of the meat 56 is to be started (step 33). The determination can be made using measurements from the sensors, such as by measuring the density of the cells of the material 56, the rate that the cells of the material 56 are decreasing nutrient concentration in the medium 58 or the concentration of waste products in the medium 58, though other ways to determine that detachment needs to be initiated are possible. Alternatively, the start of the detachment can be triggered based on another event. For example, the detachment can be started as soon as soon as the material 56 is determined to have reached a desired size based on optical sensors. Still other events that can trigger the start of the detachment are possible.

Initial parameters for a field applied to cause the detachment is determined (step 34) based on one or more of the characteristics or classification (if known) of the cells of the material 56 (or the structures within the material 56), the metasurface 62, the container 55, 56, or medium 58. The field can include a magnetic field, electric field, an electromagnetic field, or a combination of fields. Other types of fields are possible.

Meanwhile, the field parameters can include amplitude, frequency, phase, waveform, and duration, as well as other types of parameters. Values for the parameters can be determined using a look up table, which can provide field parameter values for particular combinations of particular cells of the material 56 and metasurface 62, and, if used for the determination, the characteristics and classification (if available) or the container 55, 59 and the medium 58. In a further embodiment, machine learning can be used to determine the initial field parameters. The learning can be performed based on data sets of the characteristic values and parameters for fields to be applied to each of the different objects. Once the parameters are determined, the field is then applied (step 35) to the metasurface 62, based on the values of the parameters.

To maintain desired progression towards desired cellular detachment, a monitoring-based feedback loop (steps 36-41) is run (step 36). While undergoing application of the field, the cells, metasurface 62, and optionally the container 55, 59, the medium 58, and the field can be monitored (step 36) continuously or at predetermined time periods to determine their characteristics. For example, temperature of portions of the metasurface 62 can be monitored to make sure the temperature does not rise above a predetermined threshold that could be damaging to the cells of the cells of the material 56. Similarly, temperature of the container 55, 59 and the medium 58 could be monitored. Concentration and presence of chemicals, such as nutrients and waste products, within the medium 58 can similarly be monitored. Likewise, the behavior of the cells can be monitored, including their position, which can be used to determine whether they have started detaching from the metasurface 56 to the desired extent and whether the detachment is forming desired structures. Parameters of the applied field can also be monitored, including wavelength, frequency, phase, amplitude, waveforms, and duration.

If at any time, field application becomes unnecessary (step 38), such as the desired degree of detachment has been achieved based on the data from the sensors, or if a user commands to stop application of the field, monitoring of the field ends, stopping (step 39) the feedback loop and stopping the method 30.

However, if the application of the field does not become unnecessary, the monitored characteristics can be used to determine whether the field needs to be adjusted (step 40). If the progression towards the desired goal is satisfactory (which can be determined based on predetermined benchmarks to which the measured characteristics and parameters can be compared), no adjustment may be necessary (step 39). For example, if the cells are detaching from the metasurface 62 at a desired rate as determined by an optical sensors and the temperature of the metasurface 62, the container 55, 59, and the medium is not rising above a predefined threshold, then no field parameter adjustment is necessary.

If the progression towards the desired goal is not satisfactory, the field is adjusted (step 40). For example, if the cells of the material 56 are being burned instead of detaching, the field needs to be adjusted. The field changes can be made manually or automatically. In one embodiment different formulas can be used to determine new parameter values based on the monitored characteristics and parameters, as well as a graph of characteristics and parameters and calibration of the fields. The chart can include values for the listed characteristics and parameters with standard deviations and known progression of time for each kind of meat cells and metasurface 62 with a particular characteristic or combination of characteristics to achieve the desired result. In a different embodiment, machine learning can be used to determine new values for the field parameters.

After the parameters are changed, the field is applied (step 35) to the metasurface 62 using the adjusted parameters and the feedback process continues (step 36). For example, a magnetic field can be changed by moving the magnets closer to or away from the object, or moving the magnets relative to one another. Movement of the magnets can be manual or automated.

As multiple kinds of fields can be applied to different portions of the metasurface 62, multiple feedback loops (steps 36-41) could be running at the same time. For example, as cells in corners of the meat 56 could be harder to detach than cells in other portions, a different field could be applied to the metasurface 62 on which the corners are located than inner portions of the meat 56, and the different fields could be adjusted independently of each other. Further, the field applied to one group of cells of the material 56 could be adjusted based on sensor measurements associated with another group of cells of the meat 56. For example, even if only a portion of the metasurface 62 on which the central portion of the meat 56 is located rises above a threshold temperature, the field that affects other portions of the material 56 could be adjusted to preemptively address the rising temperature.

The device 11, 27 can vary in size depending on the size of the material 56 being grown. FIG. 3 is a block diagram showing a top view of a device 11, 27 for feedback-based field application for use in the system 10 and method 30 for controlling cellular adhesion in accordance with one embodiment. The device 11, 27 can include a receptacle 70 in which the metasurface 62 with the cells is placed, either manually, via the conveyor belt 26, or through another technique. The receptacle 70 can include a container, pan, or other type of structure for holding the metasurface 62 with the cells, with the metasurface 62 possibly being a part of a container 55, 59 in which they are located. In one embodiment, the receptacle 70 is placed into a standalone housing (not shown), similar to a microwave, to initiate the field application, or alternatively, can be incorporated into another structure, such as an incubator. In a further embodiment, the receptacle 70 does not have to surround the cells from all directions.

One or more field generators 72 a,b, 73 a,b, 74 can be positioned with respect to the receptacle 70. The field generators can each include one or more magnets, one or more electrodes, both magnets and electrodes, and sources of electromagnetic field such as light sources such as halogen lamps, lasers, and light-emitting diode (LED). For example, electrodes 73 a,b can be positioned on a bottom side of the receptacle, along an interior surface, to generate a pulsed electric field. Other positions of the electrodes are possible, including on opposite sides (not shown) of the receptacle 70. When placed in a position other than the bottom of the receptacle, the electrodes can be affixed to walls of the standalone housing or walls of a housing, such as an appliance. However, at a minimum, the electrodes should be touching or at least be proximate to the cells.

The field application device 11, 27 can also include at least one magnet 72 a, b, such as an electromagnet, a permanent magnet, or a combination of magnets, to generate an oscillating magnetic, electric or electromagnetic field. Time-varying magnetic fields can be used to create electric fields and vice-versa. The magnets can be positioned along one or more sides of the receptacle 70, or can be affixed to the receptacle 70 or the housing in which the receptacle is placed. In a further embodiment, the magnets can be remotely located from the receptacle 70 and the field emitted from the magnets can be applied to the cells via one or more transducers.

Likewise, light sources 74 can be positioned anywhere from which they can deliver the light towards the portions of the metasurface 62 whose structures 57 will generate the heat necessary to trigger the detachment. The light emitted can be ultraviolet wavelength, visible light wavelength, and infrared wavelengths, though other light wavelengths are also possible.

Further, at least one closed-loop monitoring sensor 47 can be provided adjacent to the receptacle 70 on one or more sides. Alternatively or in addition, a sensor can be affixed to the housing, on an interior surface, in which the receptacle is placed for use. The monitoring sensors 71 can be active and passive, and can include reflective sensors, electric sensors, acoustic sensor, optical sensors, electrochemical sensors, thermal sensors and imagers, hyperspectral sensors, biosensors, volatile gas sensors, and sensors that are part of flow cytometry instrumentation. If sampling of the cells is necessary for analysis and by any of the sensors 71 and the sampled cells must be removed from other cells, the device 11, 27 can further include manipulators (not shown) necessary to perform the automated sampling and other pre-processing necessary for preparation of the cells for analysis by the sensors. Still other types of sensors are possible.

Different field can be applied to different groups of cells within the meat 56, such as cells located at corners of the meat 56 and central portions of the material 56. Likewise, different sensors 71 could be positioned relative to where the cells or portions of the metasurface 62, container 55, 59, or medium 58 they are intended to analyze would be located. The location of the field generators 72a,b, 73a,b, 74 and the sensors 71 could be fixed within the device 11, 27, or could be adjustable, either automatically, with machinery inside the device 11, 27 that is being controlled based on user commands, or by the user manually.

An electrical control unit 75 can be a processor that is interfaced to the sensors 71, magnets 72 a,b, and electrodes 73 a,b to communicate during the feedback process. Specifically, the processor can determine an identity of or classify cells of the material 56, metasurface 56, container 55, 59, and the medium 58 based on measurements from the sensors 71, as well as identify parameters for the field to be applied based on the identity or classification. The processor can also instruct the sensors 71 to measure characteristics of the cells, metasurface 62, container 55, 59, and medium 58, as well as the parameters of the field, and in turn, receive the measured values as feedback for determining if new parameters of the field are needed and if so, values of the parameters. Based on the feedback from the sensors, the processor can communicate the new parameter values with the field generators 72 a,b, 73a,b, 74 to change the field applied to the metasurface 62 for maintaining progress towards the desired goals.

In a further embodiment, the processor 75 can obtain data from the sensors, electrodes, and field generators for providing, via a wireless transceiver included in the device 11, 27, to a cloud-based or dedicated server 14, 16 for determining an identity or classification of the object, determining initial parameters for the field, and identifying new field parameters for adjusting the field. When performed in on the server 14, 16, the data set of characteristics, initial parameters, and guidelines for adjusted parameters can be utilized by different users. In contrast, when the processor of the device performs such actions, the data sets are specific to that field application device 11, 27.

While the description above focuses on controlling cellular adhesion of lab-grown materials, the system 10 could also be applied for other purposes, including raw, preserved or cooked foods, blood, embryos, vaccines, probiotics, medicines, sperm, tissue samples, plant cultivars, cut flowers and other plant materials, biological samples of plants, non-biologicals, such as hydrogel materials, material that can be impacted by water absorption, such as textiles, nylons and plastic lenses and optics, fine instruments and mechanical components, heat exchangers, and fuel, as well as ice as described in commonly-assigned U.S. Patent Application, entitled “System and Method for Feedback-Based Beverage Supercooling,” Ser. No. ______, filed Jul. 28, 2022, pending; ice as described in commonly-assigned U.S. Patent Application, entitled “System and Method for Controlling Crystallized Forms of Water,” Ser. No. ______, filed Jul. 28, 2022, pending; organic items as described in commonly-assigned U.S. Patent Application, entitled “System and Method for Feedback-Based Nucleation Control,” Ser. No. ______, filed Jul. 28, 2022, pending and commonly-assigned U.S. Patent Application, entitled “Feedback-Based Device for Nucleation Control,” Ser. No. ______, filed Jul. 28, 2022, pending; agriculture as described in commonly-assigned U.S. Patent application, entitled “System and Method for Controlling Cell Functioning and Motility with the Aid of a Digital Computer,” Ser. No. ______, filed Jul. 28, 2022, pending; colloids as described in commonly-assigned U.S. Patent application, entitled “System and Method for Feedback-Based Colloid Phase Change Control,” Ser. No. ______, filed Jul. 28, 2022, pending; and food as described in commonly-assigned U.S. Patent Application, entitled “System and Method for Metamaterial Array-Based Field-Shaping,” Ser. No. ______, filed Jul. 28, 2022, pending, the disclosures of which are incorporated by reference. Further, a receptacle packaging described in commonly-assigned U.S. Patent application, entitled “An Electrode Interfacing Conductive Receptacle,” Ser. No. ______, filed Jul. 28, 2022, pending, the disclosure of which is incorporated by reference, can be used to hold the cells to which the field is being applied to prevent the cells from touching electrode contacts.

While the invention has been particularly shown and described as referenced to the embodiments thereof, those skilled in the art will understand that the foregoing and other changes in form and detail may be made therein without departing from the spirit and scope of the invention.

What is claimed is:

1. A method for controlling cellular adhesion with the aid of a digital computer, comprising:
obtaining a metasurface on which cells of an artificially-grown material are located, wherein the metasurface comprises a plurality of structures associated with resonances that have a wavelength range of 250 nm-3 microns, wherein the resonances are one of localized or non-localized;
obtaining characteristics associated with the cells at one or more spatial locations of the metasurface at multiple time points and characteristics associated with the metasurface at the spatial locations at multiple time points via one or more sensors;
determining parameters of at least one field to be applied to the metasurface via at least one field generator based on the metasurface characteristics and cell characteristics determined at the multiple time points; and
controlling application of the at least one field via the at least one field generator to at least some of the structures of the metasurface based on the parameters, wherein the cells attached to those structures disengage from the structures due to the application of the field.

2. A method according to claim 1, wherein the one or more sensors comprise one or more of electromagnetic sensors, hyperspectral imaging sensors, impedance sensors, chemical sensors, biosensors, optical sensors, acoustic sensors, electrochemical sensors, and volatile gas sensors.

3. A method according to claim 1, wherein the field comprises one or more of a magnetic, electric, and electromagnetic field.

4. A method according to claim 2, wherein the electromagnetic field comprises one or more of an ultraviolet wavelength, visible light wavelength, and infrared wavelengths.

5. A method according to claim 1, wherein the at least one field generator comprises one or more of an electrode, a magnet, wires, electromagnets, two-dimensional conductive material, organic conductive polymer, a halogen lamp, a laser, and an LED.

6. A method according to claim 1, wherein the metasurface is comprised in a container in which the cells are deposited, further comprising:
determining using the one or more sensors characteristics associated with portions of the container other than the metasurface, wherein the parameters are further determined based on the container characteristics.

7. A method according to claim 6, wherein the metasurface is at least one of coated on a surface of the container and forms an integral part of the container.

8. A method according to claim 1, wherein the metasurface is an integral part of a container in which the cellular cells are deposited, further comprising;
determining using the one or more sensors characteristics associated with portions of the container other than the metasurface at the multiple time points, wherein the parameters are further determined based on the container characteristics.

9. A method according to claim 1, wherein the metasurface comprises one or more repeating motifs.

10. A method according to claim 1, wherein the cells on the metasurface are in a liquid medium, further comprising:
determining using the one or more sensors characteristics associated with the medium at the multiple time points, wherein the parameters are further determined based on the container characteristics.

11. A method according to claim 10, wherein the application of the field is triggered based on the medium characteristics at one of the time points.

12. A method according to claim 1, wherein the artificially-grown material comprises artificially-grown food.

13. A method according to claim 12, wherein the artificially-grown food comprises one or more of artificially-grown meat and artificially-grown plant-based food.

14. A method according to claim 1, further comprising:
receiving user input, wherein the parameters are further determined based on the user input.

15. A method according to claim 1, wherein the user input comprises an identification of a portion of the material that needs to disengage from the metasurface, further comprising:
selecting those of the structures to which the at least one field is applied based on the user input.

16. A method according to claim 15, wherein the disengagement of the identified portions causes at least some of the identified portions to stack up on top of further portions of the material.

17. A method according to claim 1, wherein the structures of the metasurface form one or more motifs.

18. A method according to claim 17, wherein the motifs comprise one or more of bow-tie shaped motif, a coil-shaped motif, a half-coil shaped motif, or a ribcage-shaped motif.

19. A method according to claim 11, wherein the obtaining of the metasurface and cell characteristics and controlling the application of the at least one field by the at least one field generator is performed by a processor separate from a further processor performing a determination of the parameters, wherein the processor and the further processor a wirelessly interfaced.

20. A method according to claim 1, wherein the obtaining of the metasurface and cell characteristics, controlling the application of the at least one field, and performing the determination of the parameters is performed by a single device.
